(19) United States
(12) Patent Application Publication
(10) Pub. No.: US 2024/0368060 A1
(43) Pub. Date: Nov. 7, 2024

(54) Systems and Methods for Quenching and Removal of Solids from a Hydrocarbon Gas Reaction Product

(71) Applicant: ExxonMobil Technology and Engineering Company, Baytown, TX (US)

(72) Inventors: Carlos A. Amaya, Calgary (CA); Joshua W. Thompson, Spring, TX (US); Diego E. Pena, Tomball, TX (US); Christopher J. Fowler, Houston, TX (US); James P. Chapman, Houston, TX (US)

(21) Appl. No.: 18/644,774

(22) Filed: Apr. 24, 2024

Related U.S. Application Data
(60) Provisional application No. 63/499,503, filed on May 2, 2023.

(51) Int. Cl. C07C 7/11; B01D 47/10

(52) U.S. Cl. CPC C07C 7/11; B01D 47/10

(57) ABSTRACT

Venturi-type scrubbers may be utilized in conjunction with removing solids from and quenching a gas-phase reaction product. Methods may comprise: providing a reaction product comprising a hydrocarbon gas and solids; introducing the reaction product into a Venturi-type scrubber; introducing a scrubbing fluid into the Venturi-type scrubber, wherein the scrubbing fluid is at a lower temperature than the reaction product; producing from the Venturi-type scrubber a multi-phase product comprising a scrubbed reaction product and a spent scrubbing fluid, the scrubbed reaction product comprising the hydrocarbon gas and the spent scrubbing fluid comprising the scrubbing fluid and the solids; and separating the hydrocarbon gas from the multi-phase fluid. Systems may comprise a reactor; a Venturi-type scrubber having a first inlet fluidly connected to the reactor and a second inlet configured to receive a scrubbing fluid; and a separation tower configured to receive a multi-phase stream from the Venturi-type scrubber after solids removal.

### CROSS REFERENCE TO RELATED APPLICATIONS

This application claims the benefit of and priority to U.S. Provisional Application No. 63/499,503 filed May 2, 2023, the disclosure of which is incorporated herein by reference.

### FIELD

The present disclosure relates to processing of hydrocarbon gas reaction products and, more particularly, to quenching and removal of solids from hydrocarbon gas reaction products.

### BACKGROUND

Scrubbing, or wet scrubbing, is a widely used technique to remove solids from hydrocarbon gases. The scrubbing process may involve contacting a hydrocarbon gas stream with a scrubbing fluid to capture and remove the solids from the hydrocarbon gas stream. The captured solids may then be carried along with the spent scrubbing fluid, which may then be more readily separated from the hydrocarbon gas stream before conducting further processing thereon. Scrubbers capable of performing wet scrubbing are commonly used in many industries, including oil and gas, chemical processing, and power generation technologies, where solids within a hydrocarbon gas stream may lead to issues such as, for example, fouling, erosion, and/or corrosion of process equipment; decreased operating efficiency; and adverse impacts upon product quality.

Several classes of scrubbers are presently available for performing wet scrubbing, such as packed bed scrubbers and spray towers. Venturi-type scrubbers may also be utilized in some instances. Venturi-type scrubbers employ a relatively low pressure drop to promote a separation. The pressure drop across a Venturi-type scrubber may facilitate a separation process by increasing the velocity of a feed within the Venturi-type scrubber, thereby promoting more effective contact between the feed and a scrubbing fluid.

Additionally, quenching is also frequently utilized in many industrial processes. Quench towers, for example, may be used following catalytic or thermal cracking processes to promote cooling of a reaction product through exposure to a quench fluid, wherein the cooling may also prevent further reactions from occurring. Various quench fluids may be utilized for this purpose depending on the nature of the reaction product undergoing quenching, including both water and organic fluids. Solids entrained within the reaction product may be problematic when introduced to a quench tower and may lead to at least some of the issues noted above.

### SUMMARY

In various aspects, methods of the present disclosure comprise: providing a reaction product comprising a hydrocarbon gas and solids; introducing the reaction product into a Venturi-type scrubber; introducing a scrubbing fluid into the Venturi-type scrubber, wherein the scrubbing fluid is at a lower temperature than the reaction product; producing from the Venturi-type scrubber a multi-phase product comprising a scrubbed reaction product and a spent scrubbing fluid, the scrubbed reaction product comprising the hydrocarbon gas and the spent scrubbing fluid comprising the scrubbing fluid and the solids; and separating the hydrocarbon gas from the multi-phase fluid; wherein about 90 wt % or more of the solids in the reaction product are transferred to the spent scrubbing fluid.

In other various aspects, systems of the present disclosure comprise: a reactor having an overheads outlet; a Venturi-type scrubber comprising (i) a first inlet fluidly connected to the overheads outlet via an overhead line, (ii) a second inlet fluidly connected to a scrubbing fluid line, and (iii) an outlet for a multi-phase stream; and a separation tower configured to receive the multi-phase stream after at least partial removal of solids therefrom; wherein the scrubbing fluid line conveys a scrubbing fluid comprising a hydrocarbon to the Venturi-type scrubber.

These and other features and attributes of the disclosed systems and methods of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings. The following figures are included to illustrate certain aspects of the disclosure and should not be viewed as exclusive configurations. The subject matter disclosed is capable of considerable modifications, alterations, combinations, and equivalents in form and function, as will occur to those skilled in the art and having the benefit of this disclosure.

FIG. 1A is a diagram of a first non-limiting example of a system and method for quenching and removing solids from a gas-phase reaction product using a Venturi-type scrubber.

FIG. 1B is a diagram of a second non-limiting example of a system and method for quenching and removing solids from a gas-phase reaction product using a Venturi-type scrubber.

FIG. 2 is a diagram of a non-limiting example of suitable inlet configurations for a Venturi-type scrubber.

### DETAILED DESCRIPTION

The present disclosure relates to processing of hydrocarbon gas reaction products and, more particularly, to quenching and removal of solids from hydrocarbon gas reaction products.

Systems and methods of the present disclosure may incorporate a Venturi-type scrubber to achieve concurrent quenching and solids separation from a hydrocarbon gas reaction product prior to further processing thereof in a downstream separation tower. Hydrocarbon gas reaction products suitable for processing according to the present disclosure may contain solids (e.g., aerosolized solids) and be obtained from processes such as, for example, catalytic reactions and thermal cracking (e.g., flexicoking). The solids present within the hydrocarbon gas reaction product obtained from these processes may comprise, for example, particles of catalysts or coke, respectively. Advantageously, the systems and methods of the present disclosure may achieve solids removal from the hydrocarbon gas reaction product with a relatively low pressure drop across the Venturi-type scrubber so that upstream and downstream processing operations are not adversely impacted. The actual pressure drop achieved may, at least in part, be a consequence of the design of the particular Venturi-type scrubber that is employed. Moreover, the systems and methods of the present disclosure may operate at or near atmospheric pressure and utilize a scrubbing fluid that is hydrocarbon-based to accomplish the foregoing.

In addition to promoting solids separation, quenching of the reaction product may concurrently occur in the Venturi-type scrubber. Quenching may be achieved through providing a cooled scrubbing fluid to the Venturi-type scrubber, which may further aid in limiting the pressure drop therein. Advantageously, the scrubbing fluid or a portion thereof may be recycled to the Venturi-type scrubber after promoting quenching and/or solids separation. In addition, after removal of solids from the scrubbing fluid, a solids-free scrubbing fluid may be contacted with the hydrocarbon gas reaction product as a quench oil to provide additional quenching in the course of promoting separation of the hydrocarbon gas reaction product in a separation tower. By at least partially removing solids from the hydrocarbon gas reaction product and at least partially quenching the hydrocarbon gas reaction product using a Venturi-type scrubber, internals within the separation tower may be protected from fouling, and a decreased propensity toward the occurrence of side reactions in the separation tower may be realized.

### Definitions

As used herein, a “Venturi-type scrubber” refers to a separation device configured to receive a high-velocity gas stream and discharge a multi-phase product stream having a decreased velocity. The separation device may further receive a scrubbing fluid, and the energy of the high-velocity gas stream may atomize the scrubbing fluid for contacting the components of the gas stream for forming the multi-phase product stream to promote separation of one or more components therein.

As used herein, an “entry portion” of a Venturi-type scrubber refers to a location immediately upstream from a converging cone portion of the Venturi-type scrubber. The entry portion of a Venturi-type scrubber may have a substantially constant cross-sectional diameter.

As used herein, a “converging cone portion” of a Venturi-type scrubber refers to a conical portion of the Venturi-type scrubber immediately upstream from an intermediate portion containing a throat of the Venturi-type scrubber. The converging cone portion decreases in cross-sectional diameter between the entry portion and the intermediate portion of the Venturi-type scrubber.

As used herein, a “throat” of a Venturi-type scrubber refers to a narrowest cross-sectional portion of the Venturi-type scrubber. The throat of a Venturi-type scrubber may have a substantially constant cross-sectional diameter. The throat may be located in the intermediate portion of the Venturi-type scrubber and may constitute the entirety of the intermediate portion in some cases.

As used herein, a “diverging cone portion” of a Venturi-type scrubber refers to a conical portion of the Venturi-type scrubber immediately downstream from the intermediate portion of the Venturi-type scrubber. The diverging cone portion increases in cross-sectional diameter between the intermediate portion and an outlet portion of the Venturi-type scrubber.

As used herein, an “outlet portion” of a Venturi-type scrubber refers to a location immediately downstream from the diverging cone portion of the Venturi-type scrubber. The outlet portion of a Venturi-type scrubber may have a substantially constant cross-sectional diameter.

As used herein, “coke” refers to a carbonaceous solid byproduct of pyrolysis, cracking, or a similar chemical process.

As used herein, the term “cyclic C5 hydrocarbon” refers to hydrocarbon compounds having a 5-membered carbocylic ring including, but not limited to, cyclopentane, cyclopentene, cyclopentadiene, and any combination thereof. The term “cyclic C5 hydrocarbon” also includes alkylated or polyalkylated analogs of any of the foregoing (e.g., methylcyclopentane, methylcyclopentene, methylcyclopentadiene, pentamethylcyclopentadiene, and the like).

As used herein, the term “Cn” refers to hydrocarbon(s) having n carbon atom(s) per molecule, wherein n is a positive integer. The term “Cn−” refers to hydrocarbon(s) having less than or equal to n carbon atoms per molecule. The term “Cn+” refers to hydrocarbons having greater than or equal to n carbon atoms per molecule.

As used herein, the term “hydrocarbon” refers to a class of compounds containing hydrogen bound to carbon, and encompasses (i) saturated hydrocarbon compounds, (ii) unsaturated hydrocarbon compounds, (iii) mixtures of hydrocarbon compounds (saturated and/or unsaturated), including mixtures of hydrocarbon compounds having different values of n.

As used herein, the term “flexicoking” refers to a thermal cracking process producing delayed coking in a fluidized bed reactor when cracking a hydrocarbon feedstock.

As used herein, the terms “scrubbing” or “wet scrubbing” refer to a process in which solids are removed from a gas stream by capturing the solids in a scrubbing fluid.

### Systems and Methods Using a Venturi-Type Scrubber

Methods of the present disclosure may comprise providing a reaction product comprising a hydrocarbon gas and solids, introducing the reaction product into a Venturi-type scrubber, and introducing a scrubbing fluid into the Venturi-type scrubber, wherein the scrubbing fluid is at a lower temperature than the reaction product. Because the scrubbing fluid is at a lower temperature than the reaction product, the scrubbing fluid may lower the temperature of the reaction product to promote at least partial quenching thereof. In addition to promoting at least partial quenching, the scrubbing fluid may further facilitate separation of solids from the reaction product and aid in maintaining a low pressure drop across the Venturi-type scrubber. In promoting separation of the solids, a multi-phase product comprising a scrubbed reaction product and a spent scrubbing fluid may be produced from the Venturi-type scrubber. After exiting the Venturi-type scrubber, the hydrocarbon gas may be further separated from the multi-phase fluid. The scrubbed reaction product may comprise the hydrocarbon gas and the spent scrubbing fluid may comprise the scrubbing fluid and the solids. About 90 wt % or more of the solids in the reaction product may be transferred to the spent scrubbing fluid and a decrease in pressure across the Venturi-type scrubber between the reaction product and the multi-phase product preferably may range from about 1 psi to about 3 psi.

Systems of the present disclosure may comprise a reactor having an overheads outlet; a Venturi-type scrubber comprising (i) a first inlet fluidly connected to the overheads outlet via an overhead line, (ii) a second inlet fluidly connected to a scrubbing fluid line, and (iii) an outlet for a multi-phase stream; and a separation tower configured to receive the multi-phase stream after at least partial removal of solids therefrom. The scrubbing fluid line may convey a scrubbing fluid comprising a hydrocarbon to the Venturi-type scrubber. A decrease in pressure between the first inlet and the outlet preferably may range from about 1 psi to about 3 psi. Preferably, the scrubbing fluid provided to the Venturi-type scrubber may be cooled, such as by air or chilled water cooling. Additional details follow below.

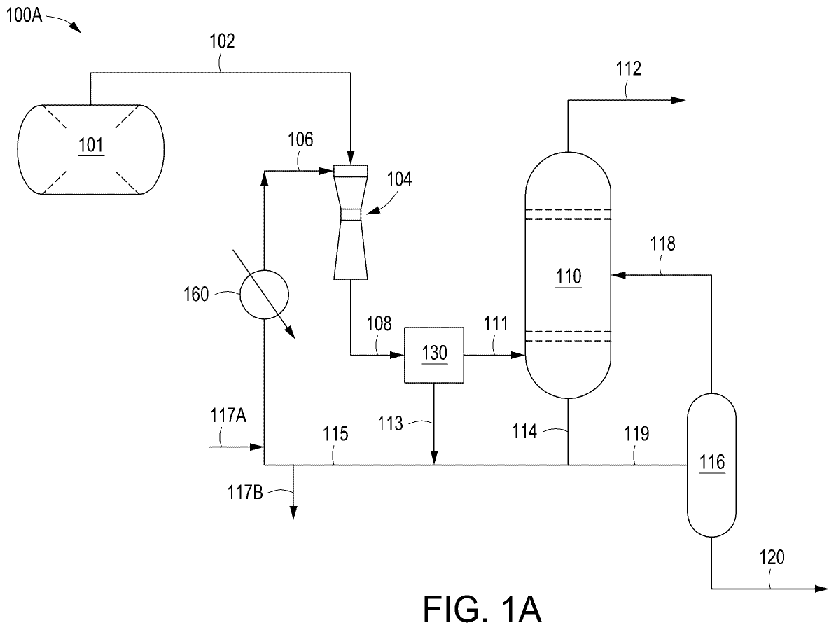

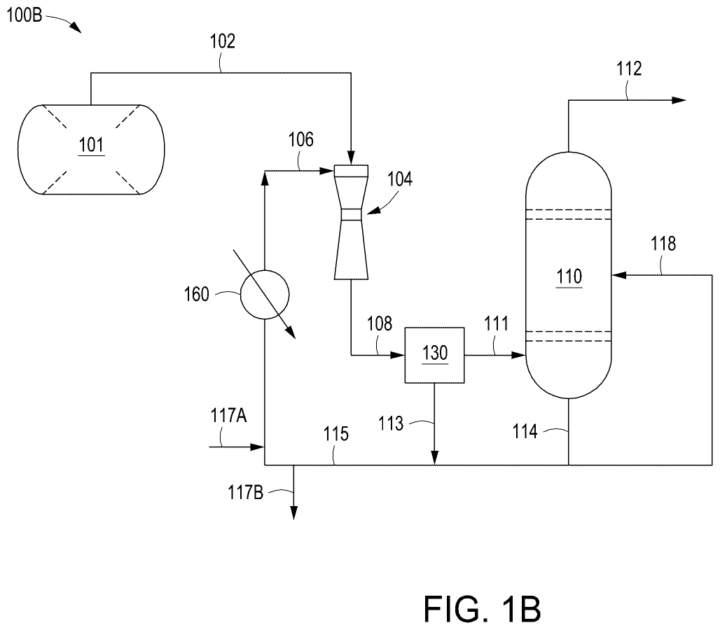

FIG. 1A is a diagram of a first non-limiting example of a system and method for quenching and removing solids from a gas-phase reaction product using a Venturi-type scrubber. System and method 100A may concurrently quench and remove solids from reaction product 102 comprising a hydrocarbon gas that is obtained from reactor 101. Reaction product 102 may be conveyed from an overheads outlet of a reactor 101 to Venturi-type scrubber 104, as described in greater detail hereinafter. Quenching and solids removal from reaction product 102 may be at least partially accomplished within Venturi-type scrubber 104. Reactor 101 may, for example, be a fluidized bed reactor, which may result in some fluidized particles (solids) exiting reactor 101 within reaction product 102.

In non-limiting examples, reactor 101 may comprise a fluidized bed reactor configured for performing a cyclization reaction, and reaction product 102 may comprise cyclic C5 hydrocarbons produced from acyclic hydrocarbons (e.g., acyclic C5 hydrocarbons, such as n-pentane) in the fluidized bed reactor. The cyclic C5 hydrocarbons may comprise one or more of cyclopentane, cyclopentene, cyclopentadiene, alkylated variants thereof, and any combination thereof. Illustrative processes and reactor systems for converting acyclic C5 hydrocarbons into cyclic C5 hydrocarbons are provided in commonly owned U.S. Patent Application Publications 20170121242, 20170121243, 20170121245, 20170121246, 20170121247, 20170121250, 20170121251, 20170121252, 20170121253, 20170121254, 20170121255, 20180319717, 20180319721, and 20180319722, each of which is incorporated herein by reference. In the interest of brevity, such processes for producing a reaction product comprising cyclic C5 hydrocarbons are only described at a high level hereinafter. A Venturi-type scrubber may be incorporated in the processes described in any of the foregoing to accomplish at least partial quenching and solids separation from the reaction product thereof according to the disclosure herein.

Catalysts suitable for converting acyclic C5 hydrocarbons into one or more cyclic C5 hydrocarbons may include microporous crystalline metallosilicate-based catalysts having a zeolite framework. Suitable microporous metallosilicates may include, but are not limited to, those of zeolite framework types MWW, MFI, LTL, MOR, BEA, TON, MTW, MTT, FER, MRE, MFS, MEL, DDR, EUO, FAU, and any combination thereof. When producing reaction product 102 containing one or more cyclic C5 hydrocarbons with the foregoing catalysts, the reaction product may have a concentration of cyclic C5 hydrocarbons of about 50 wt % or greater, or about 55 wt % or greater, or about 60 wt % or greater, or about 65 wt % or greater, or about 70 wt % or greater, or about 75 wt % or greater, or about 80 wt % or greater, or about 85 wt % or greater, or about 90 wt % or greater, each as measured relative to the mass of total hydrocarbons that have undergone conversion. Thus, the foregoing concentration values do not include hydrocarbon gases present in the feed mixture as a diluent and that do not undergo a reaction. Furthermore, solids may be entrained within reaction product 102 (i.e., catalyst particles) at a concentration of about 50 wt % or less, or about 45 wt % or less, or about 40 wt % or less, or about 35 wt % or less, or about 30 wt % or less, or about 25 wt % or less, or about 20 wt % or less, or about 15 wt % or less, or about 10 wt % or less, or about 5 wt % or less, or about 1 wt % or less, each as measured relative to the total mass of hydrocarbons and any carrier gas present (i.e., total mass of the stream). The solids may be at least partially removed from reaction product 102 through utilizing Venturi-type scrubber 104 according to the disclosure herein.

In some or other non-limiting examples, reactor 101 may, for example, be a coker, such as a delayed coker, a fluid coker, or a flexicoker, in which case the solids within reaction product 102 may comprise coke particles. The hydrocarbon gas obtained from coking may comprise low molecular weight hydrocarbons (e.g., hydrocarbons having a carbon number of 5 or less) having coke particles present therein. A concentration of coke particles in reaction product 102 may, for example, be about 50 wt % or less, or about 45 wt % or less, or about 40 wt % or less, or about 35 wt % or less, or about 30 wt % or less, or about 25 wt % or less, or about 20 wt % or less, or about 15 wt % or less, or about 10 wt % or less, or about 5 wt % or less, or about 1 wt % or less.

In still other examples, reactor 101 may similarly conduct a process in which a hydrocarbon gas reaction product is obtained with solids present therein. Examples, may include, but are not limited to, catalytic cracking, hydrocracking, chemical synthesis, gasification, the like, and any combination thereof.

Although only one Venturi-type scrubber 104 is depicted in FIG. 1A, it is to be appreciated that multiple (more than one) Venturi-type scrubber 104 may alternately be present. The multiple Venturi-type scrubbers 104 may be present in series, parallel, or any combination thereof. When multiple Venturi-type scrubbers 104 are present, the multiple Venturi-type scrubbers 104 may be of the same design/dimensions or have differing designs/dimensions. Moreover, although FIG. 1A has depicted Venturi-type scrubbers 104 in a vertical orientation, with reaction product 102 entering a top portion of Venturi-type scrubbers 104 and multi-phase product 108 exiting a bottom portion of Venturi-type scrubbers 104, it is to be appreciated that non-vertical or even horizontal orientations may also be suitable in some instances.

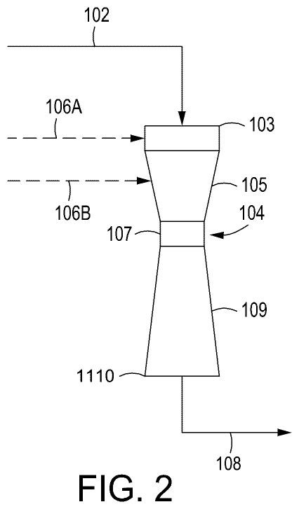

Scrubbing fluid 106 may be introduced to Venturi-type scrubber 104 at one or more inlet locations, as shown in FIG. 2. FIG. 2 is a diagram of a non-limiting example of suitable inlet configurations for Venturi-type scrubber 104. Venturi-type scrubber 104 typically includes three segments: 1) an upstream segment including entry portion 103 and converging cone portion 105, 2) an intermediate segment downstream of converging cone portion 105, including throat 107 having one end abutting a smaller end of converging cone portion 105, preferably with throat 107 having a substantially uniform cross-sectional diameter, and 3) a downstream segment including diverging cone portion 109 having a smaller end thereof abutting the other end of throat 107 and also including outlet portion 1110. Venturi-type scrubber 104 may have a longitudinal axis (not shown). When intercepted by a plane perpendicular to the longitudinal axis, the cross-sectional profile of the foregoing segments can feature various shapes including circular, square, rectangular, or other geometric shapes. The term “diameter” is used to denote the distance across the cross-sectional profile, both for circular and non-circular cross-sectional profiles.

As further shown in FIG. 2, reaction product 102 may be conveyed to entry portion 103 of Venturi-type scrubber 104, such as by injection through a round-to-rectangular transition piece (or a transition piece exhibiting a similar change in geometric shape). In one non-limiting example, scrubbing fluid 106A may also be introduced to entry portion 103 of Venturi-type scrubber 104, wherein introduction takes place downstream from the location at which reaction product 102 is introduced. For example, scrubbing fluid 106A may be introduced downstream from the round-to-rectangular transition piece. In some examples, scrubbing fluid 106A may be introduced to entry portion 103 via one or more scrubbing fluid inlets that supply scrubbing fluid 106A substantially tangential to an inner flow pathway in entry portion 103. The number of scrubbing fluid inlets providing scrubbing fluid 106A to entry portion 103 may be one, two, three, four, or even more. Furthermore, the scrubbing fluid inlets providing scrubbing fluid 106A to entry portion 103 may be shielded from direct contact with incoming reaction product 102. In some or other examples, scrubbing fluid 106B may be introduced to converging cone portion 105 of Venturi-type scrubber 104. The number of scrubbing fluid inlets providing scrubbing fluid 106B to converging cone portion 105 may be one, two, three, four, or even more. In still other examples, a first portion of scrubbing fluid 106 (e.g., scrubbing fluid 106A) may be provided to entry portion 103 of Venturi-type scrubber 104 and a second portion of scrubbing fluid 106 (e.g., scrubbing fluid 106B) may be conveyed to converging cone portion 105 of Venturi-type scrubber 104. In the instance in which separate portions of scrubbing fluid 106 are conveyed to different physical locations of Venturi-type scrubber 104 (e.g., scrubbing fluid 106A and scrubbing fluid 106B), a volume ratio of the first portion of the scrubbing fluid (e.g., scrubbing fluid 106A) to the second portion of the scrubbing fluid (e.g., scrubbing fluid 106B) may, for example, be about 1:99 to about 99:1, or about 1:99 to about 75:25, or about 1:99 to about 50:50, or about 1:99 to about 25:75, or about 25:75 to about 99:1, or about 25:75 to about 75:25, or about 25:75 to about 50:50, or about 50:50 to about 99:1, or about 50:50 to about 75:25, or about 75:25 to about 99:1. The compositions and/or particle content of scrubbing fluids 106A and 106B may be the same, substantially the same, or different, depending on particular operating conditions.

In FIG. 2, reaction product 102 and scrubbing fluid 106A/106B are introduced to entry portion 103 and/or converging cone portion 105 and migrate downward, with multi-phase product 108 eventually exiting outlet portion 1110 of Venturi-type scrubber 104. Again, it is to be appreciated that although Venturi-type scrubbers 104 is depicted in a vertical configuration in FIG. 2, non-vertical or even horizontal configurations also reside within the scope of the present disclosure.

A wetted-wall approach may be used for the design of Venturi-type scrubber 104. In particular, scrubbing fluid 106 (e.g., scrubbing fluid 106A and/or scrubbing fluid 106B) may be introduced to Venturi-type scrubber 104 such that the interior walls of the flow pathway within converging cone portion 105 downstream from the entry location of scrubbing fluid 106A and/or scrubbing fluid 106B become covered, preferably fully covered, with scrubbing fluid 106 (e.g., a film formed from scrubbing fluid 106). As the cone converges, the film upon the interior walls may increase in thickness and/or the particle concentration within the film may increase. The angle of the flow pathway within converging cone portion 105 of Venturi-type scrubber 104 may be designed such that reaction product 102 and scrubbing fluid 106 are distributed substantially uniformly from entry portion 103 through converging cone portion 105 and throat 107. Diverging cone portion 109 of Venturi-type scrubber 104 may expand continuously from throat 107 to a final cross-sectional diameter at outlet portion 1110. The angle of the internal flow pathway within diverging cone portion 109 may be selected to maximize the coalescence of liquid droplets downstream from throat 107 to form multi-phase product 108. Multi-phase product 108 is obtained from outlet portion 1110 and undergoes additional processing, as described further herein.

At least a portion of the solids present within reaction product 102 may be separated within Venturi-type scrubber 104 and sequestered within scrubbing fluid 106. Without being bound by theory, contact between reaction product 102 and scrubbing fluid 106 within Venturi-type scrubber 104 may result in capturing of at least a majority of the solids within liquid droplets of scrubbing fluid 106 exiting Venturi-type scrubber 104 as multi-phase product 108, thereby sequestering at least a portion of the solids from the hydrocarbon gas before further downstream processing takes place thereupon. In non-limiting examples, an amount of solids transferred from reaction product 102 to scrubbing fluid 106 within multi-phase product 108 may be about 90 wt % or more, or 91 wt % or more, or about 92 wt % or more, or about 93 wt % or more, or about 94 wt % or more, or about 95 wt % or more, or about 96 wt % or more, or about 97 wt % or more, or about 98 wt % or more, or about 99 wt % or more. In some examples, substantially all of the solids within reaction product 102 may be transferred to scrubbing fluid 106 within multi-phase product 108.

Scrubbing fluids 106 suitable for capturing solids present within reaction product 102 may be selected based upon the nature of the process taking place in reactor 101. In the case of cyclic C5 hydrocarbons, suitable scrubbing fluids 106 may comprise one or more hydrocarbons having a carbon number of at least 7. Examples of suitable scrubbing fluids 106 that are hydrocarbons include, but are not limited to, tetralin, heavy naphtha, heavy aromatics, diesel, the like, and any combination thereof. When reaction product 102 comprises coke particles (e.g., during flexicoking), sour water may be suitable as scrubbing fluid 106, since it is usually readily available during such processes.

To facilitate separation of solids therefrom, reaction product 102 may be introduced into Venturi-type scrubber 104 at a velocity sufficient to maintain adequate contact between reaction product 102 and scrubbing fluid 106. For example, when reaction product 102 comprises one or more cyclic C5 hydrocarbons and the solids comprise catalyst particles, reaction product 102 may reach a velocity in throat 107 of Venturi-type scrubber 104 of about 175 ft/s to about 225 ft/s, or about 175 ft/s to about 215 ft/s, or about 175 ft/s to about 205 ft/s, or about 175 ft/s to about 195 ft/s, or about 175 ft/s to about 185 ft/s, or about 185 ft/s to about 225 ft/s, or about 185 ft/s to about 215 ft/s, or about 185 ft/s to about 205 ft/s, or about 185 ft/s to about 195 ft/s, or about 195 ft/s to about 225 ft/s, or about 195 ft/s to about 215 ft/s, or about 195 ft/s to about 205 ft/s, or about 205 ft/s to about 225 ft/s, or about 205 ft/s to about 215 ft/s, or about 215 ft/s to about 225 ft/s. In the instance when reaction product 102 comprises coke particles, reaction product 102 may, for example, reach a velocity in throat 107 of Venturi-type scrubber 104 of about 375 ft/s to about 425 ft/s, or about 375 ft/s to about 415 ft/s, or about 375 ft/s to about 405 ft/s, or about 375 ft/s to about 395 ft/s, or about 375 ft/s to about 385 ft/s, or about 385 ft/s to about 425 ft/s, or about 385 ft/s to about 415 ft/s, or about 385 ft/s to about 405 ft/s, or about 385 ft/s to about 395 ft/s, or about 395 ft/s to about 425 ft/s, or about 395 ft/s to about 415 ft/s, or about 395 ft/s to about 405 ft/s, or about 405 ft/s to about 425 ft/s, or about 405 ft/s to about 415 ft/s, or about 415 ft/s to about 425 ft/s.

In the disclosure herein, at least partial removal of the solids present within reaction product 102 may be achieved with a low pressure drop across Venturi-type scrubber 104. In non-limiting examples, a decrease in pressure across Venturi-type scrubber 104 between entering reaction product 102 and exiting multi-phase product 108 may be about 3 psi or less, or about 2 psi or less, or about 1 psi or less, such as within a range from about 1 psi to about 3 psi, or about 1 psi to about 2.5 psi, or about 1 psi to about 2 psi, or about 1 psi to about 1.5 psi, or about 1.5 psi to about 3 psi, or about 1.5 psi to about 2.5 psi, or about 1.5 psi to about 2 psi, or about 2 psi to about 3 psi, or about 2 psi to about 2.5 psi, or about 2.5 psi to about 3 psi.

To facilitate the low pressure drop, scrubbing fluid 106 may be introduced to Venturi-type scrubber 104 at a temperature lower than that of reaction product 102. Moreover, in having the temperature of scrubbing fluid 106 lower than that of reaction product 102, effective quenching of reaction product 102 may take place in conjunction with at least partial removal of solids therefrom. That is, contacting scrubbing fluid 106 with reaction product 102 in Venturi-type scrubber 104 may simultaneously at least partially quench reaction product 102 by lowering the temperature thereof while also promoting separation of solids therefrom. Optionally, scrubbing fluid 106 may pass through one or more heat exchangers 160 prior to entering Venturi-type scrubber 104 in order to achieve a suitably low temperature for contacting reaction product 102 therein. In non-limiting examples, scrubbing fluid 106 may have a temperature ranging from about 40° C. to about 150° C. or about 60° C. to about 110° C., such as about 100° C., when entering Venturi-type scrubber 104.

In a first set of non-limiting examples, when reaction product 102 comprises one or more cyclic C5 hydrocarbons, reaction product 102 may be obtained from reactor 101 and introduced into Venturi-type scrubber 104 at a temperature of about 550° C. to about 600° C., or about 550° C. to about 590° C., or about 550° C. to about 580° C., or about 550° C. to about 570° C., or about 550° C. to about 560° C., or about 560° C. to about 600° C., or about 560° C. to about 590° C., or about 560° C. to about 580° C., or about 560° C. to about 570° C., or about 570° C. to about 600° C., or about 570° C. to about 590° C., or about 570° C. to about 580° C., or about 580° C. to about 600° C., or about 580° C. to about 590° C., or about 590° C. to about 600° C. In this case, the difference in temperature between reaction product 102 and multi-phase product 108 exiting Venturi-type scrubber 104 may be about 410° C. to about 450° C., or about 410° C. to about 440° C., or about 410° C. to about 430° C., or about 410° C. to about 420° C., or about 420° C. to about 450° C., or about 420° C. to about 440° C., or about 420° C. to about 430° C., or about 430° C. to about 450° C., or about 430° C. to about 440° C., or about 440° C. to about 450° C.

In a second set of non-limiting examples, when reaction product 102 comprises coke particles, reaction product 102 may be obtained from reactor 101 and introduced into Venturi-type scrubber 104 at a temperature ranging from about 200° C. to about 250° C., or about 200° C. to about 240° C., or about 200° C. to about 230° C., or about 200° C. to about 220° C., or about 200° C. to about 210° C., or about 210° C. to about 250° C., or about 210° C. to about 240° C., or about 210° C. to about 230° C., or about 210° C. to about 220° C., or about 220° C. to about 250° C., or about 220° C. to about 240° C., or about 220° C. to about 230° C., or about 230° C. to about 250° C., or about 230° C. to about 240° C., or about 240° C. to about 250° C. In this case, the difference in temperature between reaction product 102 and multi-phase product 108 exiting Venturi-type scrubber 104 may range from about 125° C. to about 175° C., or about 125° C. to about 165° C., or about 125° C. to about 155° C., or about 125° C. to about 145° C., or about 125° C. to about 135° C., or about 135° C. to about 175° C., or about 135° C. to about 165° C., or about 135° C. to about 155° C., or about 135° C. to about 145° C., or about 145° C. to about 175° C., or about 145° C. to about 165° C., or about 145° C. to about 155° C., or about 155° C. to about 175° C., or about 155° C. to about 165° C., or about 165° C. to about 175° C.

To withstand high temperatures in the foregoing ranges and to reduce abrasion by the solids, Venturi-type scrubber 104 may be fabricated from a material that is resistant to these conditions. In one example, Venturi-type scrubber 104 may be formed from a carbon steel, a cobalt-chromium alloy (e.g., STELLITE™), a hard-facing stainless steel, the like, and any combination thereof. In some or other examples, at least a portion of the flow pathway, preferably all of the flow pathway, within Venturi-type scrubber 104 may have a refractory lining, such as a chromium carbide or AR400 (abrasion-resistant steel) lining.

Multi-phase product 108 exiting Venturi-type scrubber 104 may comprise a scrubbed reaction product comprising the hydrocarbon gas of reaction product 102 and a spent scrubbing fluid comprising scrubbing fluid 106 and at least a portion of the solids present in reaction product 102. A scrubbing fluid is considered spent herein if the scrubbing fluid contains solids (e.g., catalyst or coke particles) separated from reaction product 102 or introduced from another source. Use of the term “spent” does not necessarily imply that further solids may not become sequestered therein. That is, a spent scrubbing fluid may, in some cases, remain effective to sequester additional solids. A hydrocarbon gas stream may be separated from multi-phase product 108 downstream from Venturi-type scrubber 104. In particular, multi-phase product 108 may undergo a gas-liquid separation, a gas-solid separation, a liquid-solid separation, a gas-liquid-solid separation, or any combination thereof. The separation(s) taking place may occur concurrently, or preferably in a staged fashion depending on the type of separation being performed.

Referring again to FIG. 1A, multi-phase product 108 may be conveyed to separation tower 110, from which overhead stream 112 comprising the hydrocarbon gas is obtained as final product, and bottoms stream 114 comprising the spent scrubbing fluid and optionally solids is obtained. Additional quenching, if needed, may occur in separation tower 110, as described further herein. Multi-phase product 108 may undergo at least partial separation of the scrubbed reaction product and spent scrubbing fluid/solids, either upstream from separation tower 110 or in the course of being introduced to separation tower 110. As depicted in FIG. 1A, multi-phase product 108 may optionally undergo at least partial separation in post-scrubber separator 130, and scrubbed reaction product 111 may then be provided to separation tower 110. Post-scrubber separator 130 is not a Venturi-type scrubber and is capable of at least partially separating multi-phase product 108 into scrubbed reaction product 111 having no or a substantially decreased quantity of solids and mixture 113 comprising spent scrubbing fluid and solids. Mixture 113 may comprise, for example, from 40 wt %, 45 wt %, 50 wt %, 55 wt %, 60 wt %, 65 wt %, 70 wt, or 75 wt %, to 80 wt %, 85 wt %, 90 wt %, 95 wt %, 98 wt %, or 100 wt % of the solids contained in multi-phase product 108, based on the total weight of the solids in multi-phase product 108. In non-limiting examples, post-scrubber separator 130 may feature a hydrocyclone, separation drum, settling tank, the like or any combination thereof. Mixture 113 may be obtained from post-scrubber separator 130 and recirculated to Venturi-type scrubber 104 via recycle line 115, described further herein, optionally after being further separated to remove at least a portion of the solids therefrom prior to recirculation. Hydrocyclones may be particularly advantageous in this regard, since hydrocyclones may separate at least a portion of the solids from mixture 113 in the course of also separating scrubbed reaction product 111, so that scrubbing fluid 106 being recirculated to Venturi-type scrubber 104 has a decreased solids content (is at least partially regenerated). Although shown as a separate component in FIG. 1A, post-scrubber separator 130 may alternately be connected to separation tower 110 (e.g., at the bottom of separation tower 110) or be an integral portion of separation tower 110. Alternately, post-scrubber separator 130 may be omitted, and multi-phase product 108 may be conveyed directly to separation tower 110, wherein the inlet(s) of separation tower 110 are configured to promote disengagement of the scrubbed reaction product and spent scrubbing fluid/solids from one another to facilitate separation of product in overhead stream 112 and spent scrubbing fluid and solids in bottoms stream 114. By disengaging multi-phase product 108 upon entry to separation tower 110 (or separating multi-phase product 108 upstream from separation tower 110), the scrubbed reaction product may migrate upwardly in separation tower 110 and the solids (if present) may migrate downwardly as bottoms stream 114 without significantly interacting with internal components of separation tower 110.

If needed, separation tower 110 may facilitate further quenching downstream from Venturi-type scrubber 104, as well as promote separation of the spent scrubbing fluid and solids from the final product (e.g., if at least partial separation of spent scrubbing fluid and solids has not already taken place in post-scrubber separator 130 or if the separation is incomplete). The further quenching is discussed in more detail below. Separation tower 110 may contain equipment such as one or more trays/plates, baffles, pump arounds, packing (random, structured, and/or grid), or any combination thereof to facilitate separation of overhead stream 112 from bottoms stream 114. Overhead stream 112 contains the final product, and bottoms stream 114 contains spent scrubbing fluid and solids (if not already completely removed via optional post-scrubber separator 130). If solids are being introduced to separation tower 110, multi-phase product 108 may be introduced below column packing and other internals of separation tower 110, such that bottoms stream 114 may readily collect in the column bottoms and the hydrocarbon gas may percolate through the packing and other internals before subsequently exiting as overhead stream 112.

In addition to promoting further quenching and separation of spent scrubbing fluid and optionally solids within bottoms stream 114, separation tower 110 may further promote fractionation of the scrubbed reaction product into one or more fractions based on boiling point separation. Fractions separated based upon boiling point separation may exit separation tower 110 at various locations, including as overhead stream 112 or as one or more side streams (not shown). Components of separation tower 110 and operation thereof to promote fractionation of the scrubbed reaction product will be familiar to one having ordinary skill in the art.

A pressure drop between multi-phase product 108 and bottoms stream 114 within separation tower 110 may be relatively low. For example, the pressure drop between multi-phase product 108 and bottoms stream 114 may be about 0.5 psig to about 2 psig, or about 0.5 psig to about 1.5 psig, or about 0.5 psig to about 1 psig, or about 1 psig to about 2 psig, or about 1 psig to about 1.5 psig, or about 1.5 psig to about 2 psig.

A portion of bottoms stream 114 may be recycled via recycle line 115 to Venturi-type scrubber 104 as at least a portion of scrubbing fluid 106, optionally after removing at least a portion of the solids therefrom. The spent scrubbing fluid recycled to Venturi-type scrubber 104 via recycle line 115 may be introduced as scrubbing fluid 106A, 106B, or any combination thereof. Alternately, at least a portion of mixture 113 (or an at least partially regenerated variant thereof), containing spent scrubbing fluid and solids obtained from post-scrubber separator 130, may enter recycle line 115 and be re-introduced to Venturi-type scrubber 104.

Recycle line 115 may further include heat exchanger 160, which may cool scrubbing fluid 106 to a desired temperature prior to introduction to Venturi-type scrubber 104. Illustrative temperatures for scrubbing fluid 106 being introduced to Venturi-type scrubber 104 are provided above. Heat exchanger 160 may promote cooling by chilled water cooling, air cooling, refrigeration, or any combination thereof.

Optionally, makeup scrubbing fluid 117A may be introduced to recycle line 115, intermittently or continuously, should additional scrubbing fluid 106 be needed and/or if the solids content within the spent scrubbing fluid needs to be decreased to promote effective solids separation within Venturi-type scrubber 104. It is to be appreciated that the depicted location at which makeup scrubbing fluid 117A is introduced is illustrative in nature. For example, makeup scrubbing fluid 117A may be introduced upstream or downstream from heat exchanger 160. In another example, makeup scrubbing fluid 117A may be introduced directly to Venturi-type scrubber 104.

In addition, or as an alternative to introducing makeup scrubbing fluid 117A, purge line 117B may remove at least a portion of the spent scrubbing fluid or solids being recycled to Venturi-type scrubber 104. Removal of spent scrubbing fluid via purge line 117B accompanied by introduction of makeup scrubbing fluid 117A may similarly allow the solids content in recycle line 115 to be maintained within a range suitable for promoting quenching and separation in Venturi-type scrubber 104. In still another alternative, an additional scrubber (not shown) may receive mixture 113 from post-scrubber separator 130 (or bottoms stream 114 from separation tower 110) and remove at least a portion of the solids therefrom prior to the scrubbing fluid entering recycle line 115.

A non-recycle portion of bottoms stream 114 may be conveyed to bottoms separation unit 116 via line 119. A volume ratio of the portion of bottoms stream 114 recycled to Venturi-type scrubber 104 via recycle line 115 to the non-recycle portion of bottoms stream 114 conveyed to bottoms separation unit 116 via line 119 may, for example, range from about 1:99 to about 99:1, or about 1:99 to about 75:25, or about 1:99 to about 50:50, or about 1:99 to about 25:75, or about 25:75 to about 99:1, or about 25:75 to about 75:25, or about 25:75 to about 50:50, or about 50:50 to about 99:1, or about 50:50 to about 75:25, or about 75:25 to about 99:1. A portion of mixture 113 obtained from post-scrubber separator 130 may similarly be provided to bottoms separation unit 116 for further separation as well.

Bottoms separation unit 116 may facilitate a liquid-solid separation of bottoms stream 114 (optionally in combination with mixture 113). In one example, bottoms separation unit 116 may be configured to recover solids-free scrubbing fluid as a liquid phase, in which case bottoms separation unit 116 may obtain solids-free scrubbing fluid phase 118 as an upper layer and slurry phase 120 comprising residual scrubbing fluid and solids as a lower layer. In various configurations of this type, bottoms separation unit 116 may comprise, for example, a vertical phase separator, a horizontal phase separator, a filtration unit, or any like unit. Hydrocyclones may achieve separation in a similar manner, but with providing discrete layer separation. In another example, bottoms separation unit 116 may be configured to recover solids-free scrubbing fluid as a vapor phase, in which case bottoms separation unit 116 may comprise a distillation column. With bottoms separation unit 116 comprising a distillation column, solids-free scrubbing fluid phase 118 may be obtained as an overhead stream and slurry phase 120 comprising residual scrubbing fluid and solids may be obtained as a bottoms stream. As shown, slurry phase 120 may be removed from system and method 100A upon exiting bottoms separation unit 116.

Solids-free scrubbing fluid phase 118 exiting bottoms separation unit 116 may be re-introduced to separation tower 110 as a quench oil, optionally after undergoing condensation to a liquid phase. The quench oil may promote further quenching within separation tower 110 by lowering the temperature of the hydrocarbon gas therein. In non-limiting examples, the quench oil may be introduced to separation tower 110 at a location above that at which multi-phase product 108 is introduced. In more specific examples, the quench oil may be introduced above or within the column packing or other internals within separation tower 110. Because the quench oil is substantially solids free, the quench oil may be introduced into the column packing or other internals without promoting fouling thereof. Quench oil introduced to separation tower 110 may percolate downward therein and be recovered within bottoms stream 114, wherein bottoms stream 114 may be further processed as described above.

Although not explicitly shown in FIG. 1A, at least a portion of solids-free scrubbing fluid phase 118 may be optionally recirculated to a location upstream from separation tower 110. For example, solids-free scrubbing fluid phase 118 may be provided as at least a portion of makeup scrubbing fluid 117A and/or may be introduced directly to recycle line 115 separately to provide at least a portion of scrubbing fluid 106 (i.e., scrubbing fluid 106A and/or 106B) that is being introduced to Venturi-type scrubber 104.

In the event that solids separation takes place upstream from separation tower 110 (e.g., post-scrubber separator 130 is present), bottoms stream 114 may already be substantially solids-free upon exiting separation tower 110. In the event that bottoms stream 114 is obtained in a substantially solids-free state, bottoms separation unit 116 may be optionally omitted. Should bottoms separation unit 116 be omitted, at least a portion of bottoms stream 114 may be directly recirculated to separation tower 110 as a quench oil and/or at least a portion of bottoms stream 114 may be directly introduced to recycle line 115. Such a configuration omitting bottoms separation unit 116 is shown for system and method 100B in FIG. 1B.

The present disclosure further relates to the following non-limiting embodiments:

A1. A method comprising:
providing a reaction product comprising a hydrocarbon gas and solids;
introducing the reaction product into a Venturi-type scrubber;
introducing a scrubbing fluid into the Venturi-type scrubber, wherein the scrubbing fluid is at a lower temperature than the reaction product;
producing from the Venturi-type scrubber a multi-phase product comprising a scrubbed reaction product and a spent scrubbing fluid, the scrubbed reaction product comprising the hydrocarbon gas and the spent scrubbing fluid comprising the scrubbing fluid and the solids; and
separating the hydrocarbon gas from the multi-phase fluid;
wherein about 90 wt % or more of the solids in the reaction product are transferred to the spent scrubbing fluid.

A2. The method of A1, wherein the reaction product and the scrubbing fluid are introduced to the Venturi-type scrubber at an entry portion of the Venturi-type scrubber.

A3. The method of A1 or A2, wherein the reaction product is introduced to the Venturi-type scrubber at an entry portion of the Venturi-type scrubber, and the scrubbing fluid is introduced at a converging cone portion of the Venturi-type scrubber.

A4. The method of any one of A1-A3, wherein the reaction product and a first portion of the scrubbing fluid are introduced to the Venturi-type scrubber at an entry portion of the Venturi-type scrubber, and a second portion of the scrubbing fluid is introduced to the Venturi-type scrubber at a converging cone portion of the Venturi-type scrubber.

A5. The method of A4, wherein a volume ratio of the first portion of the scrubbing fluid to the second portion of the scrubbing fluid ranges from 1:99 to 99:1.

A6. The method of any one of A1-A5, wherein the hydrocarbon gas comprises one or more cyclic C5 hydrocarbons.

A7. The method of A6, wherein a decrease in temperature across the Venturi-type scrubber between the reaction product and the multi-phase product ranges from about 410° C. to about 450° C.

A8. The method of any one of A1-A7, wherein the Venturi-type scrubber has a refractory lining.

A9. The method of any one of A1-A8, wherein the reaction product reaches a velocity of about 175 ft/s to about 225 ft/s in a throat of the Venturi-type scrubber.

A10. The method of any one of A1-A9, wherein the solids comprise catalyst particles.

A11. The method of any one of A1-A10, wherein the scrubbing fluid comprises tetralin, heavy naphtha, diesel, one or more hydrocarbons having a carbon number of at least 7, or any combination thereof.

A12. The method of any one of A1-A11, further comprising:
conveying the multi-phase product to a separation tower;
separating the multi-phase product in the separation tower to obtain an overhead stream comprising the hydrocarbon gas and a bottoms stream comprising the spent scrubbing fluid and the solids; and
recycling at least a portion of the bottoms stream to the Venturi-type scrubber as at least a portion of the scrubbing fluid.

A13. The method of A12, further comprising:
conveying a non-recycle portion of the bottoms stream to a bottoms separation unit;
separating the non-recycle portion of the bottoms stream into a solids-free scrubbing fluid phase and a slurry phase comprising the solids; and
introducing at least a portion of the solids-free scrubbing fluid phase to the separation tower as a quench oil.

A14. The method of any one of A1-A11, further comprising:
introducing the multi-phase product into a post-scrubber separator upstream from a separation tower;
obtaining from the post-scrubber separator a scrubbed reaction product having at least a decreased quantity of solids relative to the multi-phase product;
conveying the scrubbed reaction product to the separation tower;
separating the scrubbed reaction product in the separation tower to obtain an overhead stream comprising the hydrocarbon gas and a bottoms stream comprising spent scrubbing fluid and optionally, the solids; and
recycling at least a portion of the bottoms stream to the Venturi-type scrubber as at least a portion of the scrubbing fluid, and/or, if free of solids, introducing at least a portion of the bottoms stream directly to the separation tower as a quench oil.

A15. The method of A14, further comprising:
if not free of solids, conveying a non-recycle portion of the bottoms stream to a bottoms separation unit;
separating the non-recycle portion of the bottoms stream into a solids-free scrubbing fluid phase and a slurry phase comprising the solids; and
introducing at least a portion of the solids-free scrubbing fluid phase to the separation tower as a quench oil.

A16. The method of any one of A1-A5, wherein the hydrocarbon gas comprises one or more hydrocarbons having a carbon number of 5 or less.

A17. The method of any one of A1-A5 or A16, wherein a decrease in temperature across the Venturi-type scrubber between the reaction product and the multi-phase product ranges from about 125° C. to about 175° C.

A18. The method of any one of A1-A5, A16 or A17, wherein the reaction product reaches a velocity of about 375 ft/s to about 425 ft/s in a throat of the Venturi-type scrubber.

A19. The method of any one of A1-A5 or A16-A18, wherein the solids comprise coke.

A20. The method of A19, wherein the scrubbing fluid comprises sour water.

A21. The method of any one of A1-A5 or A16-A20, further comprising:
conveying the multi-phase product to a separation tower;
separating the multi-phase product in the separation tower to obtain an overhead stream comprising the hydrocarbon gas and a bottoms stream comprising the spent scrubbing fluid and the solids; and
recycling at least a portion of the bottoms stream to the Venturi-type scrubber as at least a portion of the scrubbing fluid.

A22. The method of A21, further comprising:
conveying a non-recycle portion of the bottoms stream to a bottoms separation unit;
separating the non-recycle portion of the bottoms stream into a solids-free scrubbing fluid phase and a slurry phase comprising the solids; and
introducing at least a portion of the solids-free scrubbing fluid phase to the separation tower as a quench oil.

A23. The method of any one of A1-A5 or A16-A20, further comprising:
introducing the multi-phase product into a post-scrubber separator upstream from a separation tower;
obtaining from the post-scrubber separator a scrubbed reaction product having at least a decreased quantity of solids relative to the multi-phase product;
conveying the scrubbed reaction product to the separation tower;
separating the scrubbed reaction product in the separation tower to obtain an overhead stream comprising the hydrocarbon gas and a bottoms stream comprising spent scrubbing fluid and optionally, solids; and
recycling at least a portion of the bottoms stream to the Venturi-type scrubber as at least a portion of the scrubbing fluid, and/or, if free of solids, introducing at least a portion of the bottoms stream directly to the separation tower as a quench oil.

A24. The method of A23, further comprising:
if not free of solids, conveying a non-recycle portion of the bottoms stream to a bottoms separation unit;
separating the non-recycle portion of the bottoms stream into a solids-free scrubbing fluid phase and a slurry phase comprising the solids; and
introducing at least a portion of the solids-free scrubbing fluid phase to the separation tower as a quench oil.

B1. A system comprising:
a reactor having an overheads outlet;
a Venturi-type scrubber comprising (i) a first inlet fluidly connected to the overheads outlet via an overhead line, (ii) a second inlet fluidly connected to a scrubbing fluid line, and (iii) an outlet for a multi-phase stream; and
a separation tower configured to receive the multi-phase stream after at least partial removal of solids therefrom;
wherein the scrubbing fluid line conveys a scrubbing fluid comprising a hydrocarbon to the Venturi-type scrubber.

B2. The system of B1, wherein the first inlet and the second inlet of the Venturi-type scrubber are at an entry portion of the Venturi-type scrubber.

B3. The system of B1 or B2, wherein the first inlet of the Venturi-type scrubber is at an entry portion of the Venturi-type scrubber, and the second inlet of the Venturi-type scrubber is at a converging cone portion of the Venturi-type scrubber.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present inventions. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

The present invention is well adapted to attain the ends and advantages mentioned as well as those that are inherent therein. The particular examples and configurations disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

The invention claimed is:

1. A method comprising:
providing a reaction product comprising a hydrocarbon gas and solids;
introducing the reaction product into a Venturi-type scrubber;
introducing a scrubbing fluid into the Venturi-type scrubber, wherein the scrubbing fluid is at a lower temperature than the reaction product;
producing from the Venturi-type scrubber a multi-phase product comprising a scrubbed reaction product and a spent scrubbing fluid, the scrubbed reaction product comprising the hydrocarbon gas and the spent scrubbing fluid comprising the scrubbing fluid and the solids; and
separating the hydrocarbon gas from the multi-phase fluid;
wherein about 90 wt % or more of the solids in the reaction product are transferred to the spent scrubbing fluid.

2. The method of claim 1, wherein the reaction product and the scrubbing fluid are introduced to the Venturi-type scrubber at an entry portion of the Venturi-type scrubber.

3. The method of claim 1, wherein the reaction product is introduced to the Venturi-type scrubber at an entry portion of the Venturi-type scrubber, and the scrubbing fluid is introduced at a converging cone portion of the Venturi-type scrubber.

4. The method of claim 1, wherein the reaction product and a first portion of the scrubbing fluid are introduced to the Venturi-type scrubber at an entry portion of the Venturi-type scrubber, and a second portion of the scrubbing fluid is introduced to the Venturi-type scrubber at a converging cone portion of the Venturi-type scrubber.

5. The method of claim 4, wherein a volume ratio of the first portion of the scrubbing fluid to the second portion of the scrubbing fluid ranges from 1:99 to 99:1.

6. The method of claim 1, wherein the hydrocarbon gas comprises one or more cyclic C5 hydrocarbons.

7. The method of claim 6, wherein a decrease in temperature across the Venturi-type scrubber between the reaction product and the multi-phase product ranges from about 410° C. to about 450° C.

8. The method of claim 6, wherein the reaction product reaches a velocity of about 175 ft/s to about 225 ft/s in a throat of the Venturi-type scrubber.

9. The method of claim 6, wherein the solids comprise catalyst particles.

10. The method of claim 6, wherein the scrubbing fluid comprises tetralin, heavy naphtha, diesel, one or more hydrocarbons having a carbon number of at least 7, or any combination thereof.

11. The method of claim 6, further comprising:
conveying the multi-phase product to a separation tower;
separating the multi-phase product in the separation tower to obtain an overhead stream comprising the hydrocarbon gas and a bottoms stream comprising the spent scrubbing fluid and the solids; and
recycling at least a portion of the bottoms stream to the Venturi-type scrubber as at least a portion of the scrubbing fluid.

12. The method of claim 11, further comprising:
conveying a non-recycle portion of the bottoms stream to a bottoms separation unit;
separating the non-recycle portion of the bottoms stream into a solids-free scrubbing fluid phase and a slurry phase comprising the solids; and
introducing at least a portion of the solids-free scrubbing fluid phase to the separation tower as a quench oil.

13. The method of claim 6, further comprising:
introducing the multi-phase product into a post-scrubber separator upstream from a separation tower;
obtaining from the post-scrubber separator a scrubbed reaction product having at least a decreased quantity of solids relative to the multi-phase product;
conveying the scrubbed reaction product to the separation tower;
separating the scrubbed reaction product in the separation tower to obtain an overhead stream comprising the hydrocarbon gas and a bottoms stream comprising spent scrubbing fluid and optionally, the solids; and
recycling at least a portion of the bottoms stream to the Venturi-type scrubber as at least a portion of the scrubbing fluid, and/or, if free of solids, introducing at least a portion of the bottoms stream directly to the separation tower as a quench oil.

14. The method of claim 13, further comprising:
if not free of solids, conveying a non-recycle portion of the bottoms stream to a bottoms separation unit;
separating the non-recycle portion of the bottoms stream into a solids-free scrubbing fluid phase and a slurry phase comprising the solids; and
introducing at least a portion of the solids-free scrubbing fluid phase to the separation tower as a quench oil.

15. The method of claim 1, wherein the hydrocarbon gas comprises one or more hydrocarbons having a carbon number of 5 or less.

16. The method of claim 15, wherein the solids comprise coke.

17. The method of claim 1, further comprising:
conveying the multi-phase product to a separation tower;
separating the multi-phase product in the separation tower to obtain an overhead stream comprising the hydrocarbon gas and a bottoms stream comprising the spent scrubbing fluid and the solids; and
recycling at least a portion of the bottoms stream to the Venturi-type scrubber as at least a portion of the scrubbing fluid.

18. The method of claim 17, further comprising:
conveying a non-recycle portion of the bottoms stream to a bottoms separation unit;
separating the non-recycle portion of the bottoms stream into a solids-free scrubbing fluid phase and a slurry phase comprising the solids; and
introducing at least a portion of the solids-free scrubbing fluid phase to the separation tower as a quench oil.

19. The method of claim 1, further comprising:
introducing the multi-phase product into a post-scrubber separator upstream from a separation tower;
obtaining from the post-scrubber separator a scrubbed reaction product having at least a decreased quantity of solids relative to the multi-phase product;
conveying the scrubbed reaction product to the separation tower;
separating the scrubbed reaction product in the separation tower to obtain an overhead stream comprising the hydrocarbon gas and a bottoms stream comprising spent scrubbing fluid and optionally, the solids; and
recycling at least a portion of the bottoms stream to the Venturi-type scrubber as at least a portion of the scrubbing fluid, and/or, if free of solids, introducing at least a portion of the bottoms stream directly to the separation tower as a quench oil.

20. The method of claim 19, further comprising:
if not free of solids, conveying a non-recycle portion of the bottoms stream to a bottoms separation unit;
separating the non-recycle portion of the bottoms stream into a solids-free scrubbing fluid phase and a slurry phase comprising the solids; and
introducing at least a portion of the solids-free scrubbing fluid phase to the separation tower as a quench oil.

21. A system comprising:
a reactor having an overheads outlet;
a Venturi-type scrubber comprising (i) a first inlet fluidly connected to the overheads outlet via an overhead line, (ii) a second inlet fluidly connected to a scrubbing fluid line, and (iii) an outlet for a multi-phase stream; and
a separation tower configured to receive the multi-phase stream after at least partial removal of solids therefrom;
wherein the scrubbing fluid line conveys a scrubbing fluid comprising a hydrocarbon to the Venturi-type scrubber.

22. The system of claim 21, wherein the first inlet and the second inlet of the Venturi-type scrubber are at an entry portion of the Venturi-type scrubber.

23. The system of claim 21, wherein the first inlet of the Venturi-type scrubber is at an entry portion of the Venturi-type scrubber, and the second inlet of the Venturi-type scrubber is at a converging cone portion of the Venturi-type scrubber.
